(19) United States
(12) Patent Application Publication
(10) Pub. No.: US 2026/0355153 A1
(43) Pub. Date: Nov. 12, 2026

(54) ANESTHETIC FORMULATION

(71) Applicant: Joseph Anothony Ruggirello, Macomb, MI (US)

(72) Inventor: Joseph Anothony Ruggirello, Macomb, MI (US)

(21) Appl. No.: 19/202,668

(22) Filed: May 8, 2025

(51) Int. Cl. A61K 31/381; A61K 9/00; A61K 9/08; A61K 31/137; A61K 31/167; A61K 47/02; A61K 47/12; A61K 47/18

(52) U.S. Cl. CPC A61K 31/381; A61K 9/0019; A61K 9/08; A61K 31/137; A61K 31/167; A61K 47/02; A61K 47/12; A61K 47/183

(57) ABSTRACT

A pharmaceutical composition for local dental anesthesia comprising lidocaine hydrochloride at a concentration of 2% w/v (20 mg/mL), articaine hydrochloride at a concentration of 4% w/v (40 mg/mL), epinephrine at a concentration of 1:200,000 w/v (0.005 mg/mL), and a synergistic stabilizer system comprising glycine (0.5% w/v), L-glutamic acid-L-lysine (0.3% w/v), citric acid (0.2% w/v), lactic acid (0.15% w/v), and sodium metabisulfite (0.1% w/v). Methods for administering the composition by infiltration anesthesia, block anesthesia, and intraosseous techniques are also provided, along with a method for manufacturing the composition with enhanced stability.

### FIELD OF INVENTION

The present invention relates to the field of pharmaceutical compositions for local anesthesia. More specifically, the present invention relates to a synergistic dental anesthetic formulation comprising precise concentrations of lidocaine hydrochloride, articaine hydrochloride, and epinephrine with a novel stabilizer system.

### BACKGROUND

Local anesthetics are widely used in dentistry to provide pain control during various procedures. The ideal local anesthetic for dental use should provide rapid onset, adequate duration of action, profound anesthesia, minimal systemic toxicity, and minimal post-operative complications.

The present invention addresses the limitations of these current existing dental anesthetic formulations by providing a novel composition that combines lidocaine hydrochloride and articaine hydrochloride with an optimized concentration of epinephrine and a synergistic stabilizer system.

### SUMMARY

The present invention provides a novel dental anesthetic formulation that combines the benefits of lidocaine and articaine with an optimized concentration of epinephrine and a synergistic stabilizer system.

For operations in the fields of oral surgery and dental treatment, in particular, for tooth extraction and other dental procedures, anesthetics for local injection containing lidocaine (2-diethylamino-N-(2,6-dimethyl-phenyl)acetamide) as an active ingredient have been widely used.

Commercially available formulations typically contain 20 mg of lidocaine hydrochloride and 0.0125 mg of epinephrine per 1 ml of parenteral solution (1:80,000 concentration).

Lidocaine hydrochloride (2% w/v) with epinephrine (1:80,000 or 1:100,000) has been a standard for dental anesthesia. The addition of epinephrine, a catecholamine with vasoconstrictive properties, decreases bleeding in the field of operation by lowering blood flow and reduces the systemic absorption of the anesthetic agent, thereby maintaining high concentration of the anesthetic agent in the local tissue to achieve a prolonged local anesthetic effect.

However, when conventional agents for local anesthesia are used for dental operations such as tooth extraction, which may be completed in a short period of time (typically 5 -15 minutes), the local anesthetic effect tends to be maintained longer than required (2-3 hours). As a consequence, oral and glossal numbness may remain several hours after the operation, causing difficulties in eating and drinking. Additionally, prolonged vasoconstriction from epinephrine can potentially lead to tissue necrosis and delay wound healing, particularly in patients with compromised vascular systems.

More recently, articaine has emerged as an alternative local anesthetic for dental procedures. Articaine differs from other local anesthetic agents in that it has a higher local anesthetic efficacy and a lower systemic toxicity. It contains a thiophene ring rather than a benzene ring found in other local anesthetics, which enhances its lipid solubility and ability to diffuse through tissues.

Articaine is metabolized by nonspecific plasma esterases occurring in blood and tissue, which leads to a very rapid clearance from the blood (plasma half-life of approximately 20 minutes, compared to 90 minutes for lidocaine).

Articaine is typically formulated as a 4% solution with epinephrine at concentrations of 1:100,000 or 1:200,000.

Intraosseous injection techniques have also been developed to deliver anesthetics directly to the cancellous bone surrounding the tooth, providing more targeted anesthesia with reduced quantities of anesthetic agents.

A critical aspect of local anesthetic formulations containing epinephrine is their stability. Epinephrine is traditionally considered unstable under neutral or alkaline conditions and is readily oxidized by oxygen, resulting in a colored solution ranging from red to brown. Conventional formulations use sodium metabisulfite as an antioxidant to prevent epinephrine oxidation. However, at lower epinephrine concentrations (e.g., 1:200,000 or 0.005 mg/mL), the oxidative degradation is remarkably accelerated, and conventional antioxidants alone are considered insufficient to maintain stability.

Despite advances in dental anesthesia, there remain several limitations with current formulations:
1. Traditionally, Single-agent formulations fail to leverage the complementary properties of different anesthetics.
2. Conventional epinephrine concentrations (1:80,000) may often cause prolonged numbness.
3. Reduced epinephrine concentrations lead to stability issues due to oxidative degradation.
4. Current stabilizer systems are considered inadequate for maintaining epinephrine stability at lower concentrations.
5. Existing formulations have not been optimized for both infiltration and intraosseous techniques.

An improved anesthetic formulation that combines the benefits of both lidocaine and articaine, with optimized epinephrine concentration for appropriate duration and enhanced stability, is suitable for various dental procedures and administrative techniques.

### BRIEF DESCRIPTION OF THE DRAWINGS

FIG. 1 is a diagram illustrating the various administration methods for the dental anesthetic formulation, including infiltration, block, and intraosseous techniques.

FIG. 2 is a schematic representation of the chemical structures of lidocaine and articaine, highlighting the benzene ring in lidocaine and the thiophene ring in articaine.

FIG. 3 is a graph showing the stability enhancement of epinephrine in the novel formulation compared to conventional formulations over time.

FIG. 4 is a schematic representation of the proposed mechanism of action of the synergistic stabilizer system in preventing epinephrine oxidation.

FIG. 5 is a graph showing the onset and duration of pulpal anesthesia for the novel formulation compared to conventional lidocaine and articaine formulations.

FIG. 6 is a diagram illustrating the intraosseous injection technique using the novel formulation.

### DETAILED DESCRIPTION

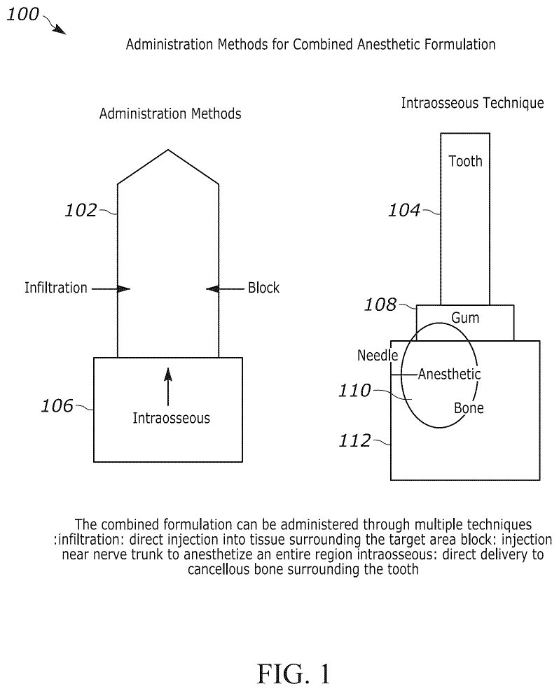

FIG. 1 demonstrates the administrative technique for the combined formulation in the anesthetic through the blocking agent and infiltration delivery vessel (102), and the intraosseous technique (106) for delivery though the tooth (104), Gum (108), and continuous delivery of the anesthetic formulation (110), past or through the bone structure (112).

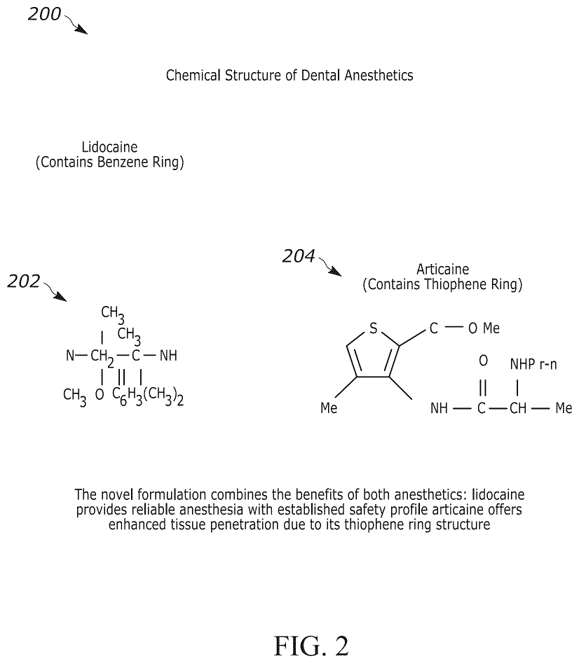

FIG. 2 demonstrates the various chemical structures for the dental anesthetics (200) which include Lidocaine (Containing the Benzene Ring) (202), and Articaine containing the Thiophene ring (204). This novel formula combines anesthesia with what the safety profile articaine would offer for enhanced tissue penetration due to the thiophene ring structure.

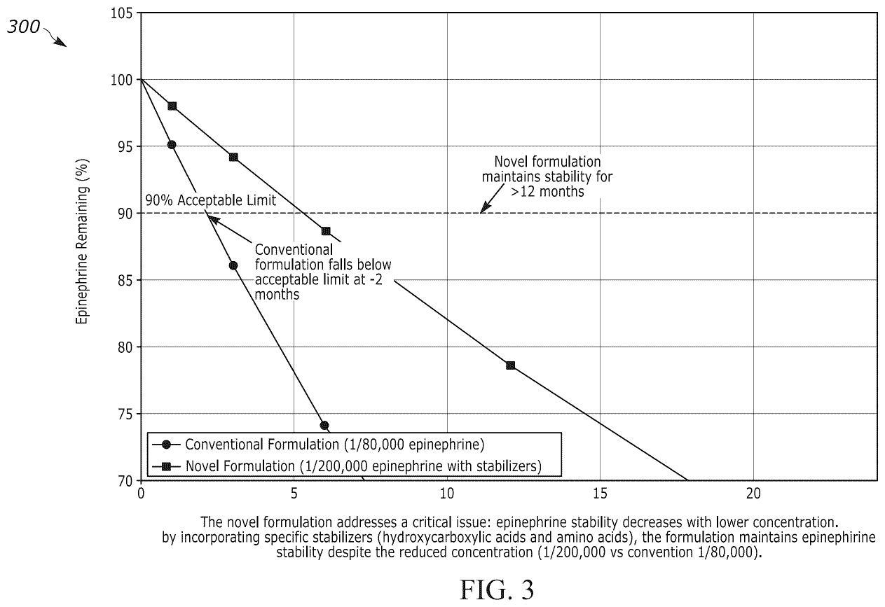

FIG. 3 represents the stability enhancement of epinephrine in the novel formulation and how the stabilizers maintain stability despite the reduced or non-conventional concentrations.

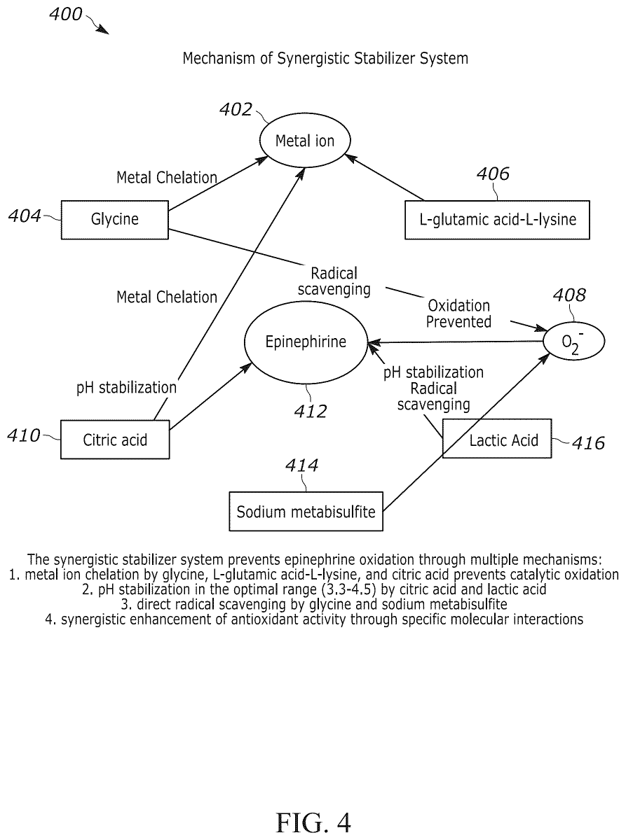

FIG. 4 represents the mechanism for the synergistic stabilizer system (400) which incorporates a metal ion in (402), Glycine in (404), L-glutamic acid-L-lysine in (406), Oxidation prevention (408), Citric Acid (410), Epinephirine (412), Lactic Acid (416) and Sodium metabisulfite (414), in which synergistic enhancement is due to the specific molecular interactions.

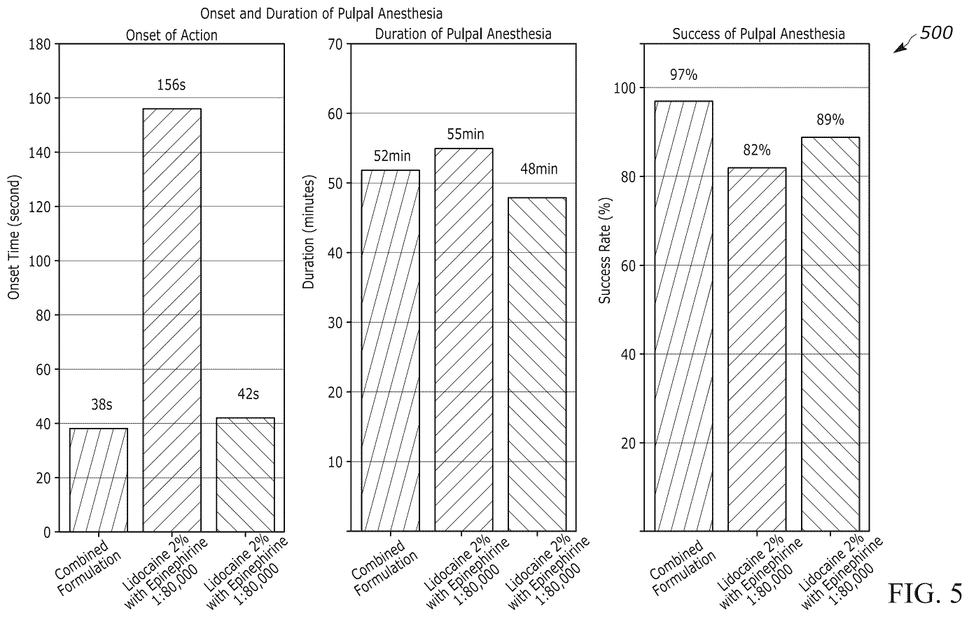

FIG. 5, which represents the onset and duration of pulpal anesthesia for the novel formulation compared to conventional lidocaine and articaine formulations is demonstrated as a graph to view onset time via the interval of seconds.

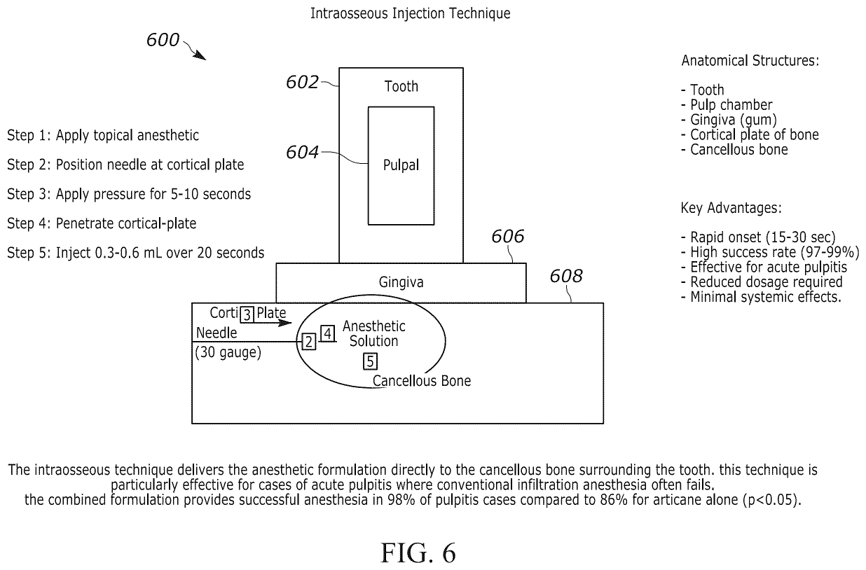

FIG. 6 represents the intraosseous injection technique in its core (600), for the tooth (602), Pulpal (604), Gingiva (606), and continued bone cavity and structure (608), it provides formulation directly to the cancellous bone surrounding the tooth. The composition in FIG. 6 for the anesthetic solution comprises the following components:
1. Active Ingredients: Lidocaine Hydrochloride: The composition contains lidocaine hydrochloride at a concentration of 2% w/v (20 mg/mL); Articaine Hydrochloride: The composition contains articaine hydrochloride at a concentration of 4% w/v (40 mg/mL); Epinephrine: The composition contains epinephrine at a concentration of 1:200,000 w/v (0.005 mg/mL as free base)
2. Stabilizer System: The composition contains a synergistic stabilizer system comprising the following components: Glycine: The composition contains glycine at a concentration of 0.5% w/v (5 mg/mL), and L-glutamic acid-L-lysine: The composition contains L-glutamic acid-L-lysine at a concentration of 0.3% w/v (3 mg/mL). This dipeptide has metal ion chelating properties and provides synergistic enhancement of antioxidant activity; Citric acid: The composition contains citric acid at a concentration of 0.2% w/v (2 mg/mL).; Lactic acid: The composition contains lactic acid at a concentration of 0.15% w/v (1.5 mg/mL); Sodium metabisulfite: The composition contains sodium metabisulfite at a concentration of 0.1% w/v (1 mg/mL).
3. Additional Excipients Sodium chloride: The composition contains sodium chloride at a concentration of 0.9% w/v (9 mg/mL) to achieve isotonicity with body fluids.
4. Water for injection: The composition contains water for injection q.s. to 1 mL as the vehicle.
5. pH adjustment: The pH of the composition is adjusted to 3.8±0.2 using sodium hydroxide or hydrochloric acid.

1. A pharmaceutical composition for local anesthesia comprising: (a) lidocaine hydrochloride at a concentration of 2% w/v (20 mg/mL); (b) articaine hydrochloride at a concentration of 4% w/v (40 mg/mL); (c) epinephrine at a concentration of 1:200,000 w/v (0.005 mg/mL); (d) a stabilizer system comprising: (i) glycine at a concentration of 0.5% w/v (5 mg/mL); (ii) L-glutamic acid-L-lysine at a concentration of 0.3% w/v (3 mg/mL); (iii) citric acid at a concentration of 0.2% w/v (2 mg/mL); (iv) lactic acid at a concentration of 0.15% w/v (1.5 mg/mL); (v) sodium metabisulfite at a concentration of 0.1% w/v (1 mg/mL); (e) sodium chloride at a concentration of 0.9% w/v (9 mg/mL); (f) water for injection q.s. to 1 mL; wherein the pH is adjusted to 3.8±0.2.

2. A method for providing local anesthesia in dental procedures, comprising administering to a patient in need thereof an effective amount of the pharmaceutical composition of claim 1.

3. The method of claim 2, wherein the pharmaceutical composition is administered by infiltration anesthesia at a dosage of 0.5-1.0 mL per site.

4. The method of claim 2, wherein the pharmaceutical composition is administered by block anesthesia at a dosage of 1.0-1.8 mL per nerve block.

5. The method of claim 2, wherein the pharmaceutical composition is administered by intraosseous technique at a dosage of 0.3-0.6 mL per site.

6. The method of claim 5, wherein the intraosseous technique comprises: (a) applying topical anesthetic to the outer epithelial layer; (b) delivering the pharmaceutical composition to the level of the cortical plate of bone in both the mesial and distal areas of a tooth with a small gauge needle; (c) applying pressure to the cortical plate of bone with the small gauge needle; and (d) either penetrating the cortical plate of bone with the small gauge needle or allowing the anesthetic to passively pass through the cortical plate of bone.

7. The method of claim 6, wherein the small gauge needle is a 30 gauge needle bent to approximately 45°.

8. A method for treating acute pulpitis, comprising administering to a patient in need thereof an effective amount of the pharmaceutical composition of claim 1 via intraosseous injection.

9. A method for manufacturing the pharmaceutical composition of claim 1, comprising: (a) preparing a solution of glycine, L-glutamic acid-L-lysine, citric acid, and lactic acid in water for injection; (b) adjusting the pH to 3.5±0.1; (c) adding sodium metabisulfite; (d) adding lidocaine hydrochloride and articaine hydrochloride; (e) separately dissolving epinephrine in water for injection containing sodium metabisulfite; (f) adding the epinephrine solution to the main solution; (g) adding sodium chloride; (h) adjusting the pH to 3.8±0.2; (i) adding water for injection to the final volume; (j) filtering the solution through a 0.22 μm sterilizing filter; (k) filling the filtered solution into dental cartridges; and (l) sterilizing the filled cartridges by autoclaving.

10. A dental kit for intraosseous dental anesthesia comprising: (a) at least one carpule of the pharmaceutical composition of claim 1; (b) at least one 30 gauge needle; and (c) instructions for use.

11. The dental kit of claim 10, further comprising a topical anesthetic.

12. The dental kit of claim 10, wherein the 30 gauge needle is pre-bent to approximately 45°.
